(19) United States
(12) Patent Application Publication
(10) Pub. No.: US 2013/0131476 A1
(43) Pub. Date: May 23, 2013

(54) OXIMETRIC PLETHYSMOGRAPHY

(71) Applicant: Oneeros, Inc., San Francisco, CA (US)

(72) Inventors: Stanley C. Siu, Castro Valley, CA (US); Arman Abdalkhani, San Francisco, CA (US)

(73) Assignee: Oneeros, Inc., San Francisco, CA (US)

(21) Appl. No.: 13/677,190

(22) Filed: Nov. 14, 2012

Related U.S. Application Data
(60) Provisional application No. 61/560,252, filed on Nov. 15, 2011.

(51) Int. Cl. A61B 5/0205; A61B 5/026; A61B 5/145; A61B 5/0295; A61B 5/1455; A61B 5/00

(52) U.S. Cl. 600/324

(57) ABSTRACT

Systems and methods for estimating a plethysmograph waveform are provided. In some aspects, a system includes a detector module configured to receive, from a single channel, an oximeter output signal indicative of light absorption in a patient. The oximeter output signal includes infrared light components and red light components. The system also includes a processing module configured to determine an indicator of a ratio of (i) an indicator of at least one of the infrared light components to (ii) an indicator of at least one of the red light components. The processing module is configured to determine, based on the indicator of the ratio, an indicator of a plethysmograph waveform of the patient.

### CROSS-REFERENCES TO RELATED APPLICATIONS

The present application claims the benefit of U.S. Provisional Patent Application Ser. No. 61/560,252, entitled “Pulse Oximetry System,” filed on Nov. 15, 2011, which is hereby incorporated by reference in its entirety for all purposes.

### FIELD

The subject technology generally relates to pulse oximetry systems and methods.

### BACKGROUND

Pulse oximetry, with heart rate detection and plethysmography, is a noninvasive procedure for measuring data points, such as during medical anesthetic and surgical cases. For example, pulse oximetry may be used to collect oxygen saturation, heart rate, and/or plethysmography data. Some of the data obtained from oximetry devices may be used to help in the diagnosis of sleep apnea. Unfortunately, as a result of sophisticated electronics associated with the oximetry devices (typically located in hospitals), many patients with sleep apnea cannot monitor their own breathing behavior at home during their sleep.

### SUMMARY

The subject technology is illustrated, for example, according to various aspects described below. Various examples of aspects of the subject technology are described as numbered clauses (1, 2, 3, etc.) for convenience. These are provided as examples, and do not limit the subject technology. It is noted that any of the dependent clauses may be combined in any combination, and placed into a respective independent clause, e.g., clauses 1, 12, and 23. The other clauses can be presented in a similar manner.

1. A system, for estimating a saturation level of oxygen in hemoglobin (SpO2), comprising:

a detector module configured to receive an oximeter output signal indicative of light absorption in a patient, the oximeter output signal alternating between infrared light components and red light components and comprising:

a first portion obtained at least partly during switching from at least one of the infrared components to at least one of the red components; and

a second portion obtained at least partly during switching from at least one of the red components to at least one of the infrared components; and

a processing module configured to estimate an SpO2 of the patient as a ratio between (i) a time derivative of the first portion and (ii) a time derivative of the second portion.

2. The system of clause 1, wherein the oximeter output signal alternates between the infrared light components and the red light components according to a predetermined frequency.

3. The system of clause 2, wherein the predetermined frequency is at least 20 hertz.

4. The system of clause 2, wherein the time derivative of the first portion is with respect to a switching time duration, and wherein the time derivative of the second portion is with respect to the switching time duration.

5. The system of clause 4, wherein the predetermined frequency is given by an inverse of the switching time duration.

6. The system of clause 1, wherein the time derivative of the first portion is from at least one of a peak, a valley, or an average of at least one of the infrared components to at least one of a peak, a valley, or an average of at least one of the red components.

7. The system of clause 1, wherein the time derivative of the second portion is from at least one of a peak, a valley, or an average of at least one of the red components to at least one of a peak, a valley, or an average of at least one of the infrared components.

8. The system of clause 1, wherein the processing module is configured to estimate the SpO2 as the ratio multiplied by a calibration factor.

9. The system of clause 1, wherein the time derivative of the first portion is a maximum derivative from at least one of the infrared components to at least one of the red components.

10. The system of clause 1, wherein the time derivative of the second portion is a minimum derivative from at least one of the red components to at least one of the infrared components.

11. The system of clause 1, wherein the at least one red components associated with the first portion is the same as the at least one red components associated with the second portion.

12. The system of clause 1, further comprising a generator module configured to generate the oximeter output signal.

13. The system of clause 12, wherein the generator module comprises:

a red light module configured to generate the red light components;

an infrared light module configured to generate the infrared light components; and

a driver configured to drive the red light module and the infrared light module such that the red light components and the infrared light components are alternately generated.

14. The system of clause 13, wherein the driver comprises a flip flop circuit.

15. The system of clause 13, wherein the driver is configured to generate a waveform signal that determines which of the red light components and the infrared light components are generated, and wherein the driver is configured to drive the red light module and the infrared light module based on the waveform signal.

16. The system of clause 15, wherein the waveform signal comprises at least one of (i) a headphone output signal from an electronic device or (ii) a stereo output signal from an electronic device.

17. A method, for estimating a saturation level of oxygen in hemoglobin (SpO2), comprising:

receiving an oximeter output signal indicative of light absorption in a patient, the oximeter output signal alternating between infrared light components and red light components and comprising:

a first portion obtained at least partly during switching from at least one of the infrared components to at least one of the red components; and

a second portion obtained at least partly during switching from at least one of the red components to at least one of the infrared components; and

estimating an SpO2 of the patient as a ratio between (i) a time derivative of the first portion and (ii) a time derivative of the second portion.

18. The method of clause 17, wherein the oximeter output signal alternates between the infrared light components and the red light components according to a predetermined frequency.

19. The method of clause 18, wherein the predetermined frequency is at least 20 hertz.

20. The method of clause 18, wherein the time derivative of the first portion is with respect to a switching time duration, and wherein the time derivative of the second portion is with respect to the switching time duration.

21. The method of clause 20, wherein the predetermined frequency is given by an inverse of the switching time duration.

22. The method of clause 17, wherein the time derivative of the first portion is from at least one of a peak, a valley, or an average of at least one of the infrared components to at least one of a peak, a valley, or an average of at least one of the red components.

23. The method of clause 17, wherein the time derivative of the second portion is from at least one of a peak, a valley, or an average of at least one of the red components to at least one of a peak, a valley, or an average of at least one of the infrared components.

24. The method of clause 17, wherein the SpO2 is estimated as the ratio multiplied by a calibration factor.

25. The method of clause 17, wherein the time derivative of the first portion is a maximum derivative from at least one of the infrared components to at least one of the red components.

26. The method of clause 17, wherein the time derivative of the second portion is a minimum derivative from at least one of the red components to at least one of the infrared components.

27. The method of clause 17, wherein the at least one red components associated with the first portion is the same as the at least one red components associated with the second portion.

28. The method of clause 17, further comprising generating the oximeter output signal.

29. The method of clause 28, wherein the generating comprises:

generating, by a red light module, the red light components;

generating, by an infrared light module, the infrared light components; and

driving, by a driver, the red light module and the infrared light module such that the red light components and the infrared light components are alternately generated.

30. The method of clause 29, wherein the driver comprises a flip flop circuit.

31. The method of clause 29, wherein the driving comprises:

generating a waveform signal that determines which of the red light components and the infrared light components are generated; and

driving the red light module and the infrared light module based on the waveform signal.

32. The method of clause 31, wherein the waveform signal comprises at least one of (i) a headphone output signal from an electronic device or (ii) a stereo output signal from an electronic device.

33. A machine-readable medium encoded with executable instructions for estimating a saturation level of oxygen in hemoglobin (SpO2), the instructions comprising code for:

receiving an oximeter output signal indicative of light absorption in a patient, the oximeter output signal alternating between infrared light components and red light components and comprising:

a first portion obtained at least partly during switching from at least one of the infrared components to at least one of the red components; and

a second portion obtained at least partly during switching from at least one of the red components to at least one of the infrared components; and

estimating an SpO2 of the patient as a ratio between (i) a time derivative of the first portion and (ii) a time derivative of the second portion

34. The machine-readable medium of clause 33, wherein the oximeter output signal alternates between the infrared light components and the red light components according to a predetermined frequency.

35. The machine-readable medium of clause 34, wherein the predetermined frequency is at least 20 hertz.

36. The machine-readable medium of clause 34, wherein the time derivative of the first portion is with respect to a switching time duration, and wherein the time derivative of the second portion is with respect to the switching time duration.

37. The machine-readable medium of clause 36, wherein the predetermined frequency is given by an inverse of the switching time duration.

38. The machine-readable medium of clause 33, wherein the time derivative of the first portion is from at least one of a peak, a valley, or an average of at least one of the infrared components to at least one of a peak, a valley, or an average of at least one of the red components.

39. The machine-readable medium of clause 33, wherein the time derivative of the second portion is from at least one of a peak, a valley, or an average of at least one of the red components to at least one of a peak, a valley, or an average of at least one of the infrared components.

40. The machine-readable medium of clause 33, wherein the SpO2 is estimated as the ratio multiplied by a calibration factor.

41. The machine-readable medium of clause 33, wherein the time derivative of the first portion is a maximum derivative from at least one of the infrared components to at least one of the red components.

42. The machine-readable medium of clause 33, wherein the time derivative of the second portion is a minimum derivative from at least one of the red components to at least one of the infrared components.

43. The machine-readable medium of clause 33, wherein the at least one red components associated with the first portion is the same as the at least one red components associated with the second portion.

44. The machine-readable medium of clause 33, wherein the instructions further comprise code for generating the oximeter output signal.

45. The machine-readable medium of clause 44, wherein the generating comprises:

generating, by a red light module, the red light components;

generating, by an infrared light module, the infrared light components; and

driving, by a driver, the red light module and the infrared light module such that the red light components and the infrared light components are alternately generated.

46. The machine-readable medium of clause 45, wherein the driver comprises a flip flop circuit.

47. The machine-readable medium of clause 45, wherein the driving comprises:

generating a waveform signal that determines which of the red light components and the infrared light components are generated; and

driving the red light module and the infrared light module based on the waveform signal.

48. The machine-readable medium of clause 47, wherein the waveform signal comprises at least one of (i) a headphone output signal from an electronic device or (ii) a stereo output signal from an electronic device.

49. A system, for estimating a plethysmograph waveform, comprising:

a detector module configured to receive, from a single channel, an oximeter output signal indicative of light absorption in a patient, the oximeter output signal comprising infrared light components and red light components; and

a processing module configured to determine an indicator of a ratio of (i) an indicator of at least one of the infrared light components to (ii) an indicator of at least one of the red light components,

wherein the processing module is configured to determine, based on the indicator of the ratio, an indicator of a plethysmograph waveform of the patient.

50. The system of clause 49, wherein the indicator of the at least one red light component comprises at least one of a derivative, an integral, a peak, a valley, or an average of the at least one red light component.

51. The system of clause 49, wherein the indicator of the at least one infrared light component comprises at least one of a derivative, an integral, a peak, a valley, or an average of the at least one infrared light component.

52. The system of clause 49, wherein the indicator of the ratio comprises a saturation level of oxygen in hemoglobin (SpO2) of the patient.

53. The system of clause 49, wherein the processing module is configured to estimate a heart rate of the patient based on the indicator of the ratio.

54. The system of clause 49, wherein the indicator of the plethysmograph waveform comprises at least one of a heart rate of the patient or pulsatile arterial blood flow information regarding the patient.

55. The system of clause 49, further comprising a generator module configured to generate the oximeter output signal.

56. The system of clause 55, wherein the oximeter output signal alternates between the infrared light components and the red light components.

57. The system of clause 55, wherein the generator module comprises:

a red light module configured to generate the red light components;

an infrared light module configured to generate the infrared light components; and

a driver configured to drive the red light module and the infrared light module such that the red light components and the infrared light components are alternately generated.

58. The system of clause 57, wherein the oximeter output signal comprises the alternately generated red light components and infrared light components.

59. The system of clause 57, wherein the driver is configured to generate a waveform signal that determines which of the red light components and the infrared light components are generated, and wherein the driver is configured to drive the red light module and the infrared light module based on the waveform signal.

60. The system of clause 59, wherein the waveform signal comprises at least one of (i) a headphone output signal from an electronic device or (ii) a stereo output signal from an electronic device.

61. A method, for estimating a plethysmograph waveform, comprising:

receiving, from a single channel, an oximeter output signal indicative of light absorption in a patient, the oximeter output signal comprising infrared light components and red light components;

determining an indicator of a ratio of (i) an indicator of at least one of the infrared light components to (ii) an indicator of at least one of the red light components; and

determining, based on the indicator of the ratio, an indicator of a plethysmograph waveform of the patient.

62. The method of clause 61, wherein the indicator of the at least one red light component comprises at least one of a derivative, an integral, a peak, a valley, or an average of the at least one red light component.

63. The method of clause 61, wherein the indicator of the at least one infrared light component comprises at least one of a derivative, an integral, a peak, a valley, or an average of the at least one infrared light component.

64. The method of clause 61, wherein the indicator of the ratio comprises a saturation level of oxygen in hemoglobin (SpO2) of the patient.

65. The method of clause 61, further comprising estimating a heart rate of the patient based on the indicator of the ratio.

66. The method of clause 61, wherein the indicator of the plethysmograph waveform comprises at least one of a heart rate of the patient or pulsatile arterial blood flow information regarding the patient.

67. The method of clause 61, further comprising generating the oximeter output signal.

68. The method of clause 67, wherein the oximeter output signal alternates between the infrared light components and the red light components.

69. The method of clause 67, wherein the generating comprises:

generating, by a red light module, the red light components;

generating, by an infrared light module, the infrared light components; and

driving the red light module and the infrared light module such that the red light components and the infrared light components are alternately generated.

70. The method of clause 69, wherein the oximeter output signal comprises the alternately generated red light components and infrared light components.

71. The method of clause 69, wherein the driving comprises:

generating a waveform signal that determines which of the red light components and the infrared light components are generated; and

driving the red light module and the infrared light module based on the waveform signal.

72. The method of clause 71, wherein the waveform signal comprises at least one of (i) a headphone output signal from an electronic device or (ii) a stereo output signal from an electronic device.

73. A machine-readable medium encoded with executable instructions for estimating a plethysmograph waveform, the instructions comprising code for:

receiving, from a single channel, an oximeter output signal indicative of light absorption in a patient, the oximeter output signal comprising infrared light components and red light components;

determining an indicator of a ratio of (i) an indicator of at least one of the infrared light components to (ii) an indicator of at least one of the red light components; and

determining, based on the indicator of the ratio, an indicator of a plethysmograph waveform of the patient.

74. The machine-readable medium of clause 73, wherein the indicator of the at least one red light component comprises at least one of a derivative, an integral, a peak, a valley, or an average of the at least one red light component.

75. The machine-readable medium of clause 73, wherein the indicator of the at least one infrared light component comprises at least one of a derivative, an integral, a peak, a valley, or an average of the at least one infrared light component.

76. The machine-readable medium of clause 73, wherein the indicator of the ratio comprises a saturation level of oxygen in hemoglobin (SpO2) of the patient.

77. The machine-readable medium of clause 73, wherein the instructions further comprise code for estimating a heart rate of the patient based on the indicator of the ratio.

78. The machine-readable medium of clause 73, wherein the indicator of the plethysmograph waveform comprises at least one of a heart rate of the patient or pulsatile arterial blood flow information regarding the patient.

79. The machine-readable medium of clause 73, wherein the instructions further comprise code for generating the oximeter output signal.

80. The machine-readable medium of clause 79, wherein the oximeter output signal alternates between the infrared light components and the red light components.

81. The machine-readable medium of clause 79, wherein the generating comprises:

generating, by a red light module, the red light components;

generating, by an infrared light module, the infrared light components; and

driving the red light module and the infrared light module such that the red light components and the infrared light components are alternately generated.

82. The machine-readable medium of clause 81, wherein the oximeter output signal comprises the alternately generated red light components and infrared light components.

83. The machine-readable medium of clause 81, wherein the driving comprises:

generating a waveform signal that determines which of the red light components and the infrared light components are generated; and

driving the red light module and the infrared light module based on the waveform signal.

84. The machine-readable medium of clause 83, wherein the waveform signal comprises at least one of (i) a headphone output signal from an electronic device or (ii) a stereo output signal from an electronic device.

85. A system, for estimating a plethysmograph waveform, comprising:

a detector module configured to receive, from a single channel, an oximeter output signal indicative of light absorption in a patient, the oximeter output signal comprising infrared light components and red light components; and

a processing module configured to determine, based on the oximeter output signal, an indicator of a plethysmograph waveform of the patient.

86. The system of clause 85, wherein the processing module is configured to determine an indicator of a ratio of (i) an indicator of at least one of the infrared light components to (ii) an indicator of at least one of the red light components.

87. The system of clause 86, wherein the processing module is configured to determine, based on the indicator of the ratio, the indicator of the plethysmograph waveform of the patient.

88. A method, for estimating a plethysmograph waveform, comprising:

receiving, from a single channel, an oximeter output signal indicative of light absorption in a patient, the oximeter output signal comprising infrared light components and red light components; and

determining, based on the oximeter output signal, an indicator of a plethysmograph waveform of the patient.

89. The method of clause 88, further comprising determining an indicator of a ratio of (i) an indicator of at least one of the infrared light components to (ii) an indicator of at least one of the red light components.

90. The method of clause 89, wherein the determining comprises determining, based on the indicator of the ratio, the indicator of the plethysmograph waveform of the patient.

91. A machine-readable medium encoded with executable instructions for estimating a plethysmograph waveform, the instructions comprising code for:

receiving, from a single channel, an oximeter output signal indicative of light absorption in a patient, the oximeter output signal comprising infrared light components and red light components; and

determining, based on the oximeter output signal, an indicator of a plethysmograph waveform of the patient.

92. The machine-readable medium of clause 91, wherein the instructions further comprise code for determining an indicator of a ratio of (i) an indicator of at least one of the infrared light components to (ii) an indicator of at least one of the red light components.

93. The machine-readable medium of clause 92, wherein the determining comprises determining, based on the indicator of the ratio, the indicator of the plethysmograph waveform of the patient.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 illustrates an example of pulse oximetry sensor system that comprises a sensor and a monitor.

FIG. 2 illustrates an example of an electret microphone and its interface with a mobile device.

FIG. 3 illustrates an example of using a pulsing hardware circuit, in accordance with various aspects of the subject technology.

FIG. 4 illustrates an example of circuitry that can be used as pulsing hardware, in accordance with various aspects of the subject technology.

FIG. 5 illustrates an example of using headphone/stereo output voltage to act as LED drivers, in accordance with various aspects of the subject technology.

FIG. 6 illustrates an example of a signal processing scheme to extract a red and infrared signal, and ultimately the SpO₂ signal from the oximeter signal, in accordance with various aspects of the subject technology.

FIG. 7 illustrates sample data collected with an audio oximeter setup, in accordance with various aspects of the subject technology.

FIG. 8A illustrates an example of a pulse oximeter signal output, in accordance with various aspects of the subject technology.

FIG. 8B illustrates an example of building or extracting composite red and infrared signals, in accordance with various aspects of the subject technology.

FIG. 9A illustrates an RC circuit connected to an oximeter output before connecting to an audio input port and audio processor, in accordance with various aspects of the subject technology.

FIG. 9B illustrates an oximeter square wave and a resultant differentiated signal seen by the audio processor, in accordance with various aspects of the subject technology.

FIG. 9C illustrates an example of determining SpO₂, in accordance with various aspects of the subject technology.

FIG. 10A illustrates a square wave and a resultant differentiated signal, in accordance with various aspects of the subject technology.

FIG. 10B illustrates graphs that show the calculation of slopes of the square wave, in accordance with various aspects of the subject technology.

FIGS. 11A and 11B illustrate graphs that the relationship between the red signal and the infrared signal, in accordance with various aspects of the subject technology.

FIGS. 12A and 12B illustrate an example of an alternate scheme to determine SpO₂, in accordance with various aspects of the subject technology.

FIGS. 13A and 13B illustrate another example to determine SpO₂, in accordance with various aspects of the subject technology.

FIG. 14 illustrates an example of how to calculate SpO₂, in accordance with various aspects of the subject technology.

FIG. 15 illustrates an example of a system for estimating SpO₂, in accordance with various aspects of the subject technology.

FIG. 16 illustrates an example of a method for estimating SpO₂, in accordance with various aspects of the subject technology.

FIGS. 17A and 17B illustrate an example of an oximeter output signal that may be used to determine a plethysmographic waveform of a patient, in accordance with various aspects of the subject technology.

FIG. 18 illustrates an example of a system for estimating a plethysmographic waveform, in accordance with various aspects of the subject technology.

FIG. 19 illustrates an example of a method for estimating a plethysmographic waveform, in accordance with various aspects of the subject technology.

FIG. 20 is a conceptual block diagram illustrating an example of a system, in accordance with various aspects of the subject technology.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

Pulse oximetry may rely on the different light absorption characteristics of oxygenated and unoxygenated hemoglobin. Typically, in pulse oximetry, a sensor is placed on a thin part of a patient's body, usually a fingertip or ear lobe. Red and infrared light emitting diodes (LEDs) may be alternately turned on and off (e.g., pulsed), and passed through the patient. Transmitted or reflected light may then be collected by a detector, and sophisticated electronics can be used to interpret the oximetry data. However, as a result of the sophisticated electronics (typically located in hospitals), many patients with sleep apnea cannot monitor their own breathing behavior at home during their sleep.

Aspects of the subject technology solve the foregoing problem by providing an oximetry device that can couple to an audio input port of any suitable computing device (e.g., mobile phone, laptop computer, desktop computer, tablet, etc.). The oximetry device may provide oximetry data to the computing device via the audio input port, and software on the computing device may be used to record and interpret the data. For example, a patient may use the oximetry device at home while sleeping. The oximetry device can be connected to the patient's mobile phone, which may then be able to collect oximetry data from the oximetry device and generate diagnostic information (e.g., the patient's breathing patterns) based on the oximetry data. In some aspects, the diagnostic information may be transmitted to the patient's doctor using the mobile phone (or some other suitable computing device). The use of the audio input port may offer a universal, low cost, and mobile alternative to otherwise expensive and sophisticated dedicated electronics to perform oximetry measurements.

In some embodiments, circuitry is provided to pulse the red and infrared LEDs of the oximetry device, and also to enable the connection between the oximetry device and the computing device via the audio input port. For example, this circuitry may mimic an electret microphone, which is typically used to connect to the audio input port of the computing device. In some embodiments, circuitry is provided to use the headphone/stereo output voltage from the computing device to drive (e.g., to power and/or switch) the LEDs of the oximetry device. In some embodiments, a method for estimating the saturation level of oxygen in hemoglobin (SpO₂) of a patient is provided. The method comprises receiving an oximeter output signal. The oximeter output signal may comprise a red light signal passed through the patient and an infrared light signal passed through the patient. The method may also comprise estimating the SpO₂ as a ratio of a derivative of the red light signal to a derivative of the infrared light signal.

In some embodiments, an electronic low pass filter may be used to filter the signal from an oximeter output signal. The filtered oximeter output signal may then be passed through a blocking capacitor circuit into the audio input port of a computing device. The low pass filter may integrate the oximeter output signal, and the blocking capacitor circuit may differentiate the filtered oximeter output signal, thereby restoring the original oximeter output signal.

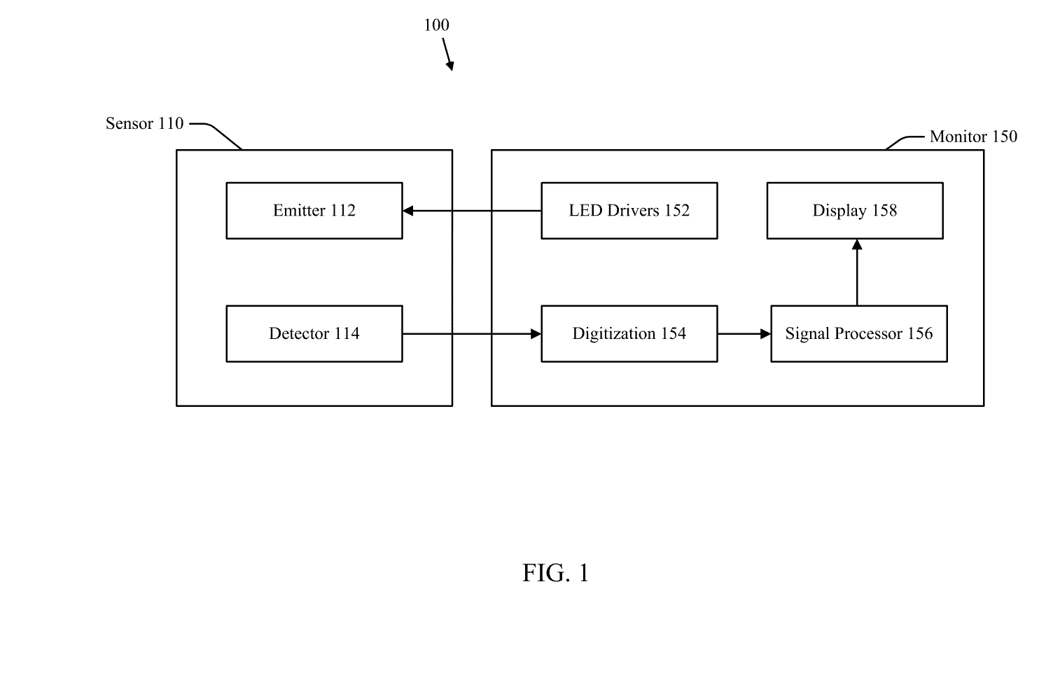

FIG. 1 illustrates an example of pulse oximetry sensor system 100 that comprises sensor 110 and monitor 150. Sensor 110, which can be attached to any number of skin surfaces such as the fingertip, earlobe, or forehead, comprises red and infrared (IR) LEDs 112 and photodiode detector 114. In the case of a finger, for example, sensor 110 is configured such that LEDs 112 project light through the fingernail and into the blood vessels and capillaries underneath. Monitor 150 comprises LED drivers 152, signal digitization 154, signal processor 156, and display 158. LED drivers 152 may alternately activate the red and IR LEDs 112, and front-end 154 may digitize the resulting current generated by photodiode 114, which may be proportional to the intensity of the detected light. Signal processor 156 may input the conditioned photodiode signal and determine oxygen saturation based on the differential absorption by arterial blood of the two wavelengths emitted by the LEDs 112. Specifically, a ratio of detected red and infrared intensities may be calculated by signal processor 156, and an arterial oxygen saturation value may be empirically determined based on the ratio obtained. Display 158 may indicate a patient's oxygen saturation, heart rate, and plethysmographic waveform.

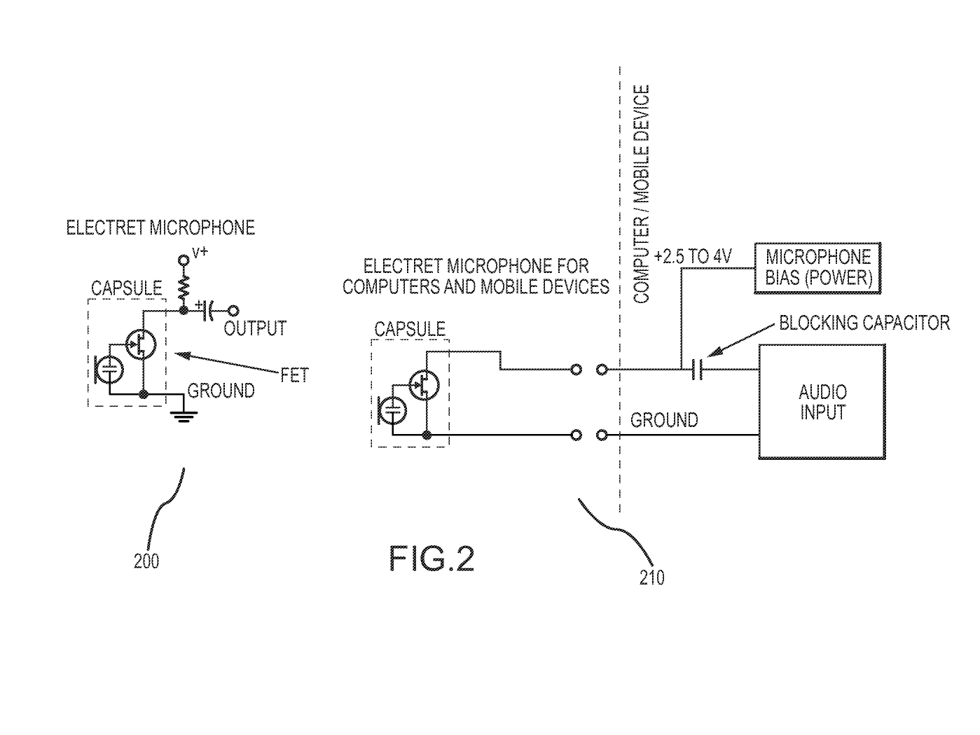

As discussed above, circuitry is provided to pulse the red and infrared LEDs of an oximetry device (e.g., oximetry sensor system 100), and also to enable the connection between the oximetry device and the computing device via the audio input port. For example, this circuitry may mimic an electret microphone. FIG. 2 illustrates an example of electret microphone 200 and its interface with mobile device 210, which can be any suitable computing device. An electret microphone preamp circuit may use a field-effect transistor (FET) in a common source configuration. The two-terminal electret capsule contains a FET that may be externally powered by supply voltage V. The resistor may set the gain and output impedance. The audio signal may appear at the output, after a direct current (DC) blocking capacitor.

Recent developments have led to the widespread use of computing devices, such as computers and digital mobile devices, that are equipped with data input ports and are designed to manage digital data. These input ports may vary widely in their design and may often be proprietary. However, many of these computing devices (e.g., cellular phones, tablet computers, music players, etc.) have audio input ports, such as analog audio input ports. According to certain aspects of the subject technology, oximetry technology may be used with the audio input ports of the computing devices to record and/or analyze oximetry data.

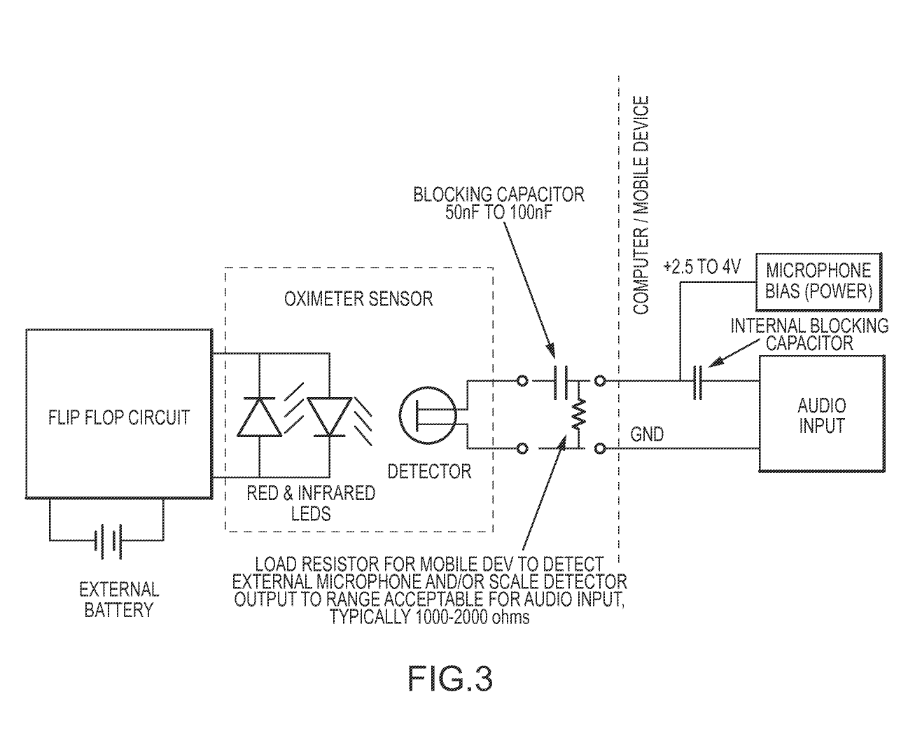

According to certain aspects of oximetry, constant signals may be emitted and captured for long segments of time. Thus, a design for an external power source may be implemented for an audio-port oximeter to assist in its ability to run. FIG. 3 illustrates an example of using a pulsing hardware circuit, which can be a flip flop circuit attached to an external battery that alternates the delivery of energy to the red and IR LEDs, in accordance with various aspects of the subject technology. This signal from the red and IR LEDs may then be captured by the sensor unit's detector. A blocking capacitor (e.g., with a value of 50 nanofarads (nF) to 100 nF, although other values greater than or less than this range may be used) and a load resistor is placed before the audio connection to eliminate the DC bias that may otherwise bias and interfere with the operation of the detector. In some aspects, to allow the mobile device (or some other suitable computing device) to detect that the audio input is being used and to modulate the detector output to a range compatible with the audio device, a load resistor with a value between 1000 ohms to 2000 ohms can be used. However, the load resistor may have other suitable values greater than or less than this range.

With the hardware configuration as illustrated in FIG. 3, the oximeter signal can be converted to a form that mimics an electret microphone and can then be recorded and subsequently processed by the computing device. The red and infrared data points as well as plethysmography data may be captured by the computing device (e.g., using hardware, software, or a combination of both). For example, using software may not require a timing circuit to distinguish the red and IR signal, as this signal may automatically provide correlation to SpO₂. Values of the blocking capacitor and load resistor may depend on the specifics of the audio input hardware. In some cases, the use of the load resistor may not be necessary.

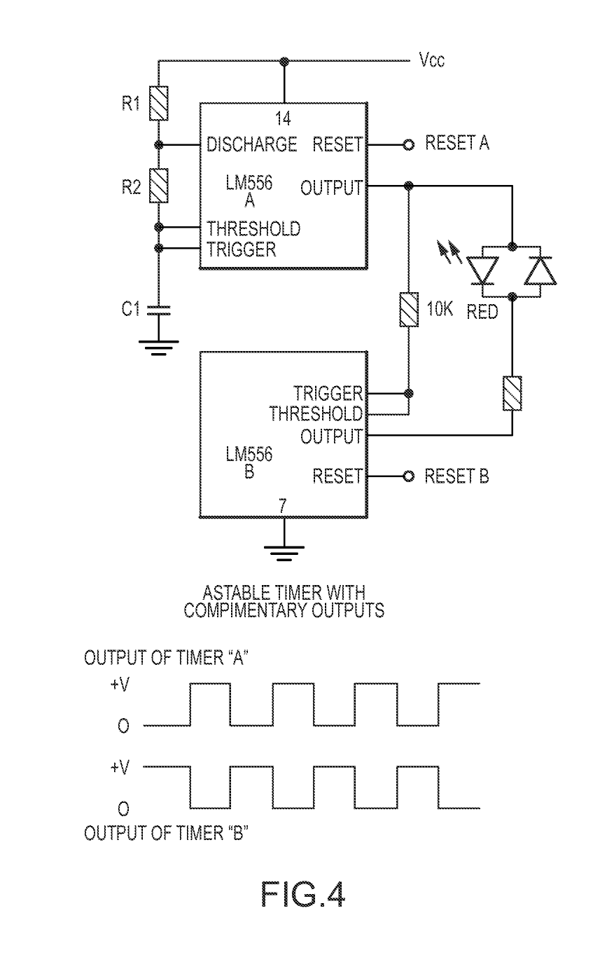

FIG. 4 illustrates an example of circuitry that can be used as pulsing hardware, in accordance with various aspects of the subject technology. In some aspects, specific configurations for this flip flop circuit may include low power timer chips running in astable mode t. The values of C1, R1, and R2 may be determined by the load cycle and frequency desired to power the LEDs.

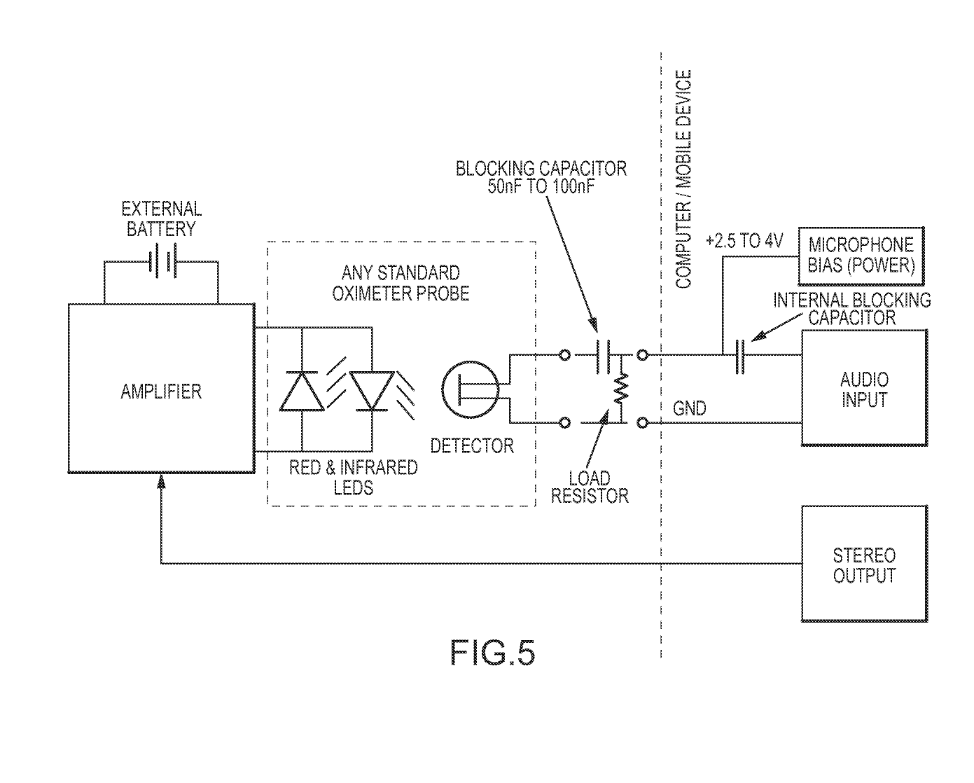

FIG. 5 illustrates an example of using headphone/stereo output voltage to act as LED drivers (e.g., drivers 152 of FIG. 1), in accordance with various aspects of the subject technology. In some aspects, an external battery source may be used for amplification, as most stereo output signals may be underpowered for this task. Use of the headphone/stereo output to determine the waveform to drive the LEDs can be used to give added capability of sending complex pulses for calibration or other purposes. For example, it may be desirable to send a set number of pulses and a set pause time (e.g., no power) to aid in the calibration of the oximeter to remove ambient light noise. The set number of pulses can also be used to aid in determining which LED (either red or IR) is activated at the time. For example, a series of three pulses to turn on the red LED followed by one pulse to turn on the IR LED may enable differentiation of the red and IR signals.

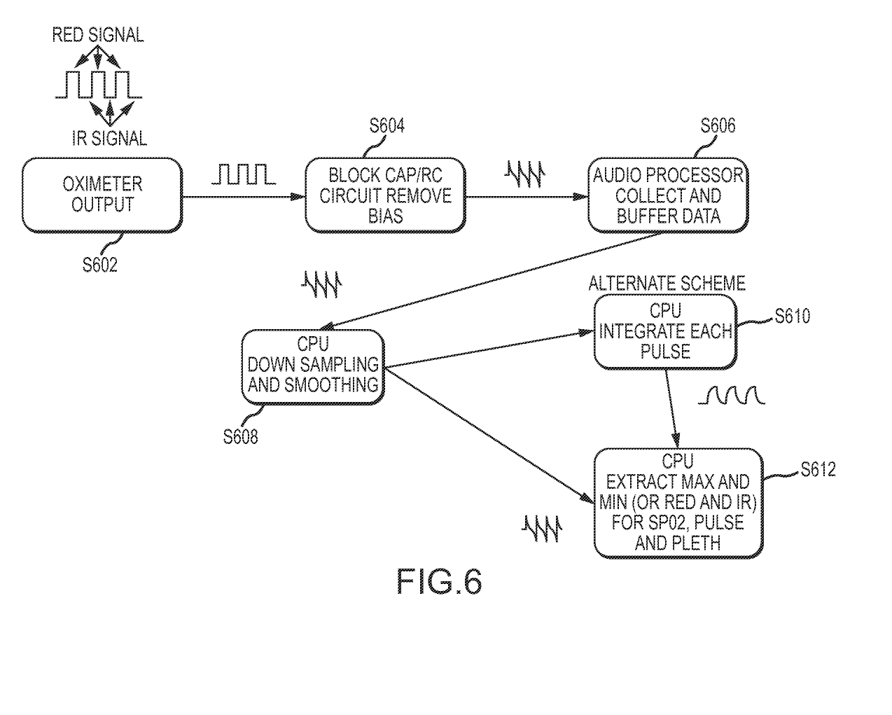

FIG. 6 shows an example of a signal processing scheme to extract the red and IR signal, and ultimately the SpO₂ signal from the oximeter signal, in accordance with various aspects of the subject technology. The signal processing scheme includes receiving the oximeter signal (S602) and sending the oximeter signal through the blocking capacitor or RC circuit (S604), which may result in applying the mathematical operation of differentiating each pulse. As each pulse may be a function of two separate and independent signals based on the red and IR response oxygen content of the hemoglobin, the result of the differentiation may be a complex function and mixture of the red and IR signals. This resultant signal may yield a signal that may be substantially identical to the SpO₂ signal. According to certain aspects, the differentiated signal may be collected and buffered (S606), and may also be down sampled and smoothed (S608). In some aspects, the differentiated signal may be directly used to calculate the SpO₂ signal (S612). In some aspects, the red and IR signals may be deconvoluted by use of numerical integration of each pulse (S610).

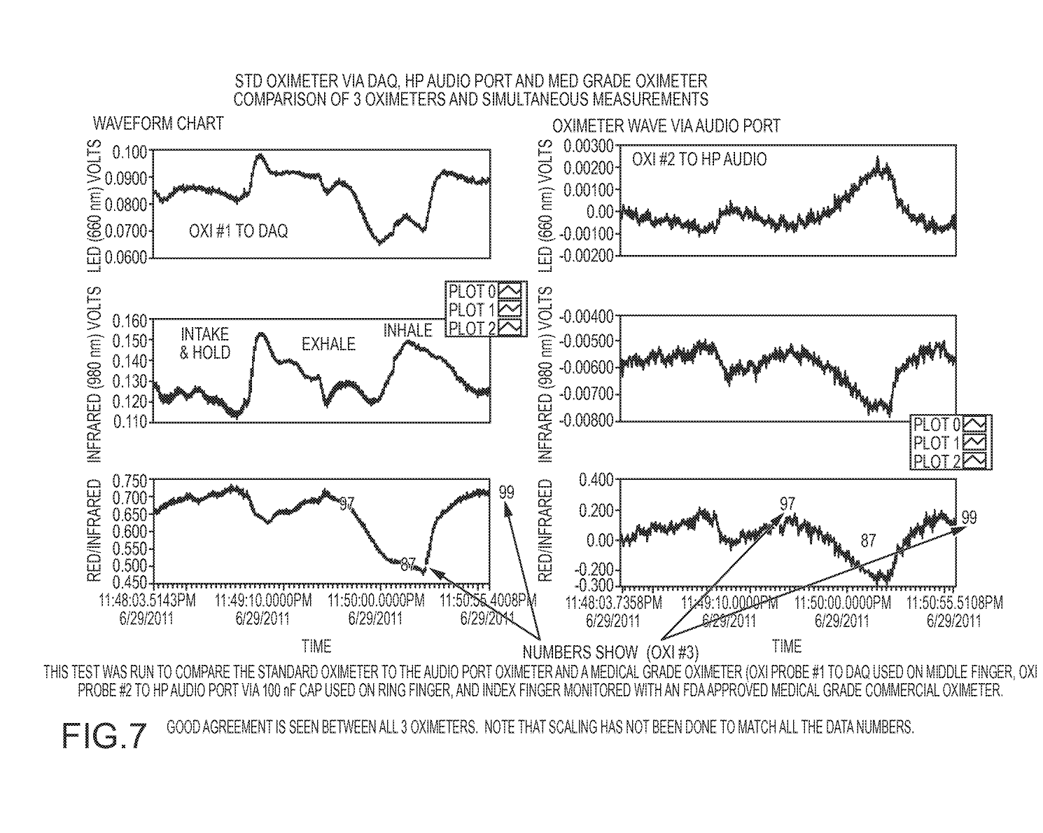

FIG. 7 illustrates sample data collected with an audio oximeter setup, in accordance with various aspects of the subject technology. The data is compared to a standard oximeter measurement, and also compared with SpO₂ numbers recorded from a medical grade oximeter. The data illustrates good agreement in SpO₂ trends between a standard oximeter and the subject technology, thereby illustrating that using the differentiated signal may yield the SpO₂ that is calculated from the separate red and IR signals typically used with a standard oximeter. The SpO₂ values from a medical grade oximeter taken simultaneously with the standard and novel device shows good agreement. It should be noted that the audio and standard oximeter numbers are not scaled, but a simple calibration can make the numbers match.

According to various aspects of the subject technology, the SpO₂ of a patient may be estimated using a derivative of the red signal and/or a derivative of the IR signal, for example, when sending the oximeter signal (e.g., which may be approximated as a square pulse) through an RC circuit to make it compatible for an audio port to process. The SpO₂ calculation may be unexpected, as the audio processing in the device may provide derivative values of the red and infrared signals (e.g., S604 in FIG. 6). According to certain aspects of the subject technology, taking the ratio of the peaks (e.g., maximums such as local maximums) of these derivatives provides proportionality to standard red/infrared ratios, and can approximate the SpO₂ after being multiplied by a constant (e.g., S612 in FIG. 6). Alternatively, the inherent derivative signal can be integrated and the resultant sinusoidal wave may approximate the raw data square wave (e.g., S610 in FIG. 6). According to certain aspects, sending the oximeter signal (e.g., approximated as a square pulse) through an RC circuit to make it compatible for an audio port to process may not be an obvious solution, since the square wave is transformed by the RC circuit. It is not obvious what part of the transformed signal should be used for determining the red and IR signals and to ultimately determine SpO₂.

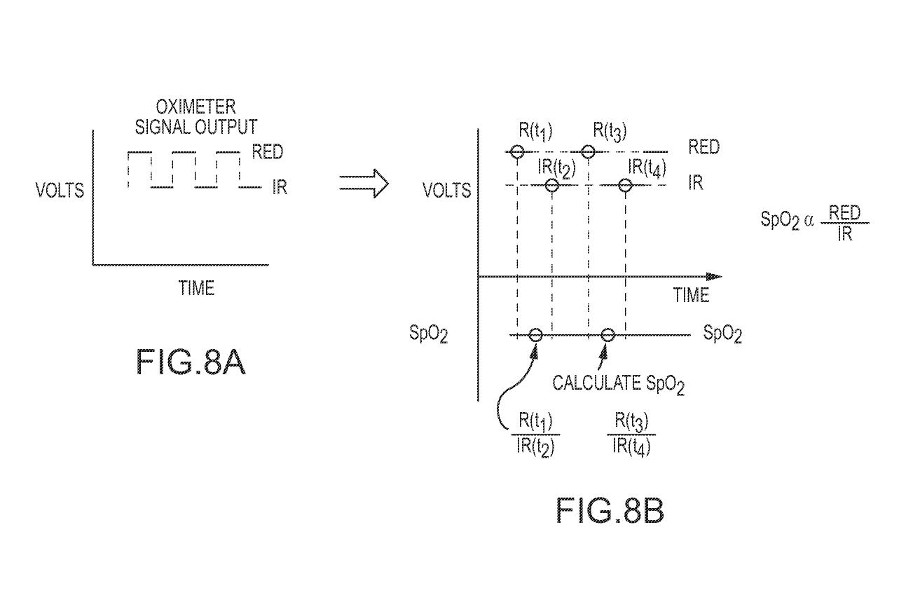

FIG. 8A illustrates an example of a typical pulse oximeter signal output from the detectors. The red and IR LEDs are alternately powered, resulting in a substantially square wave output signal from the oximeter detector. In this case, the maximum (max) may correspond to the red LED intensity and the minimum (min) of the square wave may correspond to the IR LED intensity as seen by the detector, which may convert light energy into an electrical potential. FIG. 8B illustrates that the composite red and IR signals can be built or extracted from the square wave. The ratio of the red and IR signals may be proportional to SpO₂. For example, according to certain aspects, SpO₂ may be equal to k₁+k₂*A_{red}/A_{IR}+k₃*(red/IR)̂2, where A_{red} and A_{IR} are respective absorbances of the red and IR signals, and k₁, k₂, and k₃ are calibration constants. In some aspects, A_{red} and A_{IR} may be proportional to the red and IR signals, respectively. In some aspects, SpO₂ may be proportional to a function of the ratio of the red and IR signals. For example, SpO₂ may be equal to k₁+k₂*red/IR+k₃*(red/IR)̂2+k₄(red/IR)̂3 . . . and so forth, where the k's are calibration constants. In some aspects, SpO₂ may be proportional to a function of the ratio of the derivatives of the red and IR signals (e.g., R′ and IR', respectively). For example, SpO₂ may be equal to c₁+c₂*R′/IR′+c₃*(R′/IR′)̂2+c₄(R′/IR′)̂3 . . . and so forth, where the c's are calibration constants. Since red and IR data is not collected simultaneously, but separated by the power pulsing frequency, extrapolation or approximations of the true SpO₂ can be made. In this case, if the pulsing frequency is very high, taking sequential data of red signal R(t₁) and IR signal IR(t₂) may give a fairly accurate value of SpO₂ at that time window. Similar treatment of the data may give the next value of SpO₂, where SpO₂ may be proportional to R(t₃)/IR(t₄).

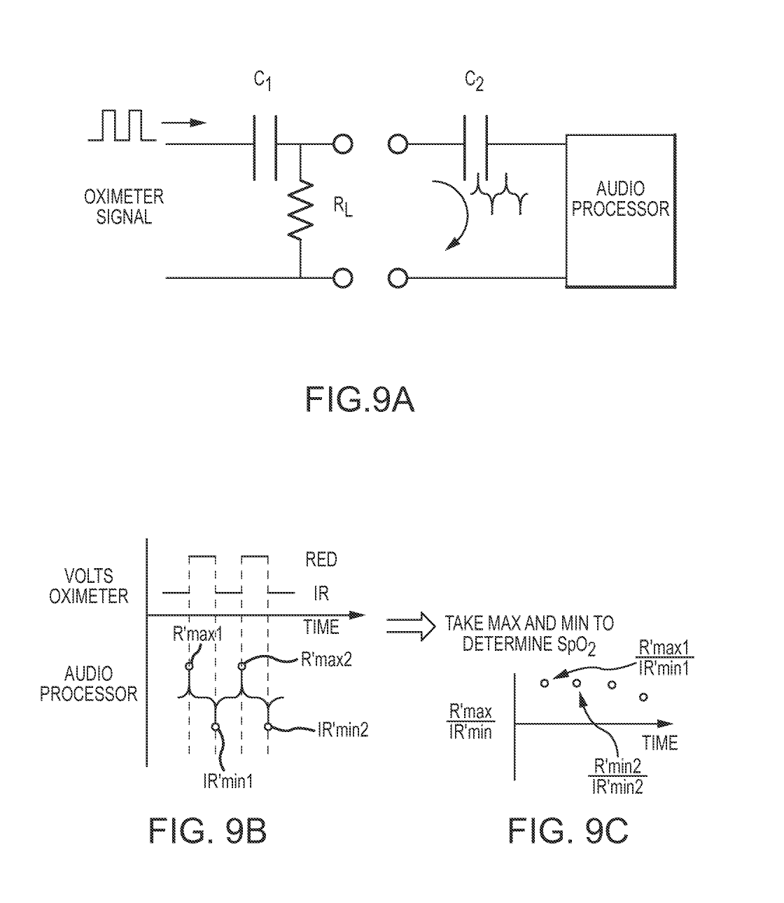

FIG. 9A illustrates the RC circuit connected to the oximeter output before connecting to the audio input port (e.g., the audio jack) and audio processor, in accordance with various aspects of the subject technology. The square wave signal from the oximeter detector may be transformed as it goes through the capacitors (e.g., C₁ and C₂). This transform may be the mathematical operation of differentiation, resulting in a “spikey signal.” It may not be obvious which part of the transformed signal may be used to determine the red signal R and the infrared signal IR to determine SpO₂.

FIG. 9B illustrates the oximeter square wave and the resultant differentiated signal seen by the audio processor, in accordance with various aspects of the subject technology. The peaks, which are circled, of the differentiated wave may correspond to the square wave edges and are labeled R′ and IR'. In some aspects, the peaks from the differentiated wave may be used to determine SpO₂ where R′ is divided by IR', as illustrated in FIG. 9C. This process may be a similar treatment to determining SpO₂ by dividing R by IR.

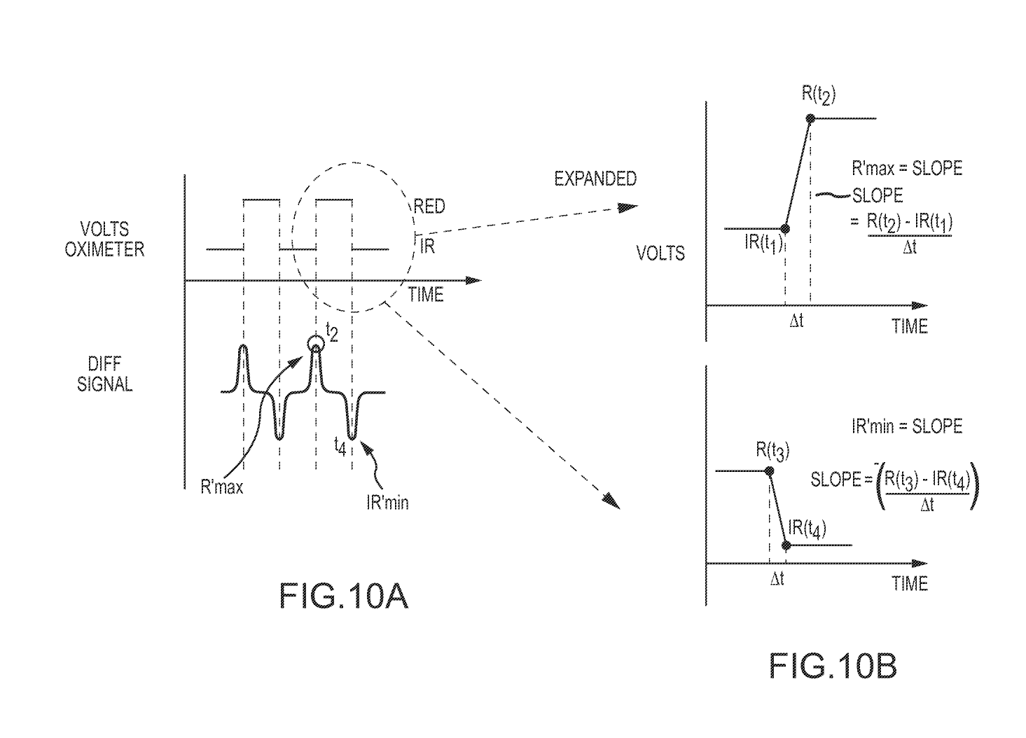

FIG. 10A illustrates the square wave and the resultant differentiated signal, in accordance with various aspects of the subject technology. FIG. 10B illustrates graphs that show the calculation of the slope at the rising and tailing edges/slopes of the square wave (or maximum and minimum of the differentiated signal), in accordance with various aspects of the subject technology. Note that theoretically, the rising and tailing edges/slopes may be functions of both R and IR. Based on the graphs of FIG. 10B, the following can be obtained:

$$\begin{matrix}\frac{{R}_{max ( {t}_{2} )}^{′}}{{IR}_{min ( {t}_{4} )}^{′}} = \frac{\frac{R ( {t}_{2} ) - IR ( {t}_{1} )}{Δ t}}{- ( \frac{R ( {t}_{3} ) - IR ( {t}_{4} )}{Δ t} )} \\ = \frac{R ( {t}_{2} ) - IR ( {t}_{1} )}{- R ( {t}_{3} ) + IR ( {t}_{4} )} \\ = \frac{R ( {t}_{2} ) - IR ( {t}_{1} )}{IR ( {t}_{4} ) - R ( {t}_{3} )}\end{matrix} \qquad ( 1 )$$

In general, suppose {IR₀, IR₁, IR₂, . . . , IRₙ₋₁} and {R₀, R₁, R₂, . . . , Rₙ₋₁} provide an initial set of data. The curve that may be observed from this data may be a polynomial of degree n that fits this given data. That is,

P(x)=a₀+a₁(x−IR₀)+a₂(x−IR₀)(x−IR₁)+a₃(x−IR₀)(x−IR₁)(x−IR₂)++aₙ(x−IR₀)(x−IR₁)(x−IR₂)(x−IR₃) . . . (x'IRₙ₋₁). (2)

In this regard, aᵢs may be found by setting

a0=R0 (3)

Then R₁=P(IR₁)=a₀+a₁(IR₁−IR₀). Now a₀=R₀ can be substituted, and therefore R₁=R₀+a₁(IR₁−IR₀), which implies

$${a}_{1} = \frac{{R}_{1} - {R}_{0}}{{IR}_{1} - {IR}_{0}} . \qquad ( 4 )$$

To find a₂, we set R₂=P(IR₂)=a₀+a₁ (IR₂−IR₀)+a₂ (IR₂−IR₀)(IR₂−IR₁), but we already have a₀ and a₁, and we can calculate a₂ as

$${a}_{2} = \frac{\frac{{R}_{2} - {R}_{1}}{{IR}_{2} - {IR}_{1}} - \frac{{R}_{1} - {R}_{0}}{{IR}_{1} - {IR}_{0}}}{{IR}_{2} - {IR}_{0}} . \qquad ( 5 )$$

To find a₃, we set R₃=P(IR₃) and so on. For the first three terms, P(x) may look like:

$$P ( x ) = {R}_{0} + \frac{{R}_{1} - {R}_{0}}{{IR}_{1} - {IR}_{0}} ( x - {IR}_{0} ) + \frac{\frac{{R}_{2} - {R}_{1}}{{IR}_{2} - {IR}_{1}} - \frac{{R}_{1} - {R}_{0}}{{IR}_{1} - {IR}_{0}}}{{IR}_{2} - {IR}_{0}} ( x - {IR}_{0} ) ( x - {IR}_{1} ) + … \qquad ( 6 )$$

This equation can be simplified and P(x) can be rewritten as:

$$P ( x ) = \frac{( x - {IR}_{1} ) ( x - {IR}_{2} ) … ( x - {IR}_{n - 1} )}{( {IR}_{0} - {IR}_{1} ) ( {IR}_{0} - {IR}_{2} ) … ( {IR}_{0} - {IR}_{n - 1} )} {R}_{0} + \frac{( x - {IR}_{0} ) ( x - {IR}_{2} ) … ( x - {IR}_{n - 1} )}{( {IR}_{1} - {IR}_{0} ) ( {IR}_{1} - {IR}_{2} ) … ( {IR}_{1} - {IR}_{n - 1} )} {R}_{1} + … \frac{( x - {IR}_{0} ) ( x - {IR}_{1} ) … ( x - {IR}_{n - 2} )}{( {IR}_{n - 1} - {IR}_{0} ) ( {IR}_{n - 1} - {IR}_{1} ) … ( {IR}_{n - 1} - {IR}_{n - 2} )} {R}_{n - 1} \qquad ( 7 )$$

Note that equation (7) may have n terms, each a polynomial of degree n−1 and each constructed in a way such that it will be zero at all of the IRᵢ except one, at which it is constructed to be Rᵢ.

The equations above (e.g., equations (1), (2), (3), (4), (5), (6), and/or (7)) show that if the max slope value R′ is divided by the min slope value IR′, the result may be a function that is a combination of R and IR, and thus, it is not obvious how to separate or isolate the terms since R and IR may be about the same.

According to certain aspects of the subject technology, experiments may show that

$$\frac{{R}_{max}^{′}}{{IR}_{min}^{′}} ∝ {SpO}_{2} ∝ \frac{R ( t )}{IR ( t )} or \frac{{R}_{max}}{{IR}_{max}}$$

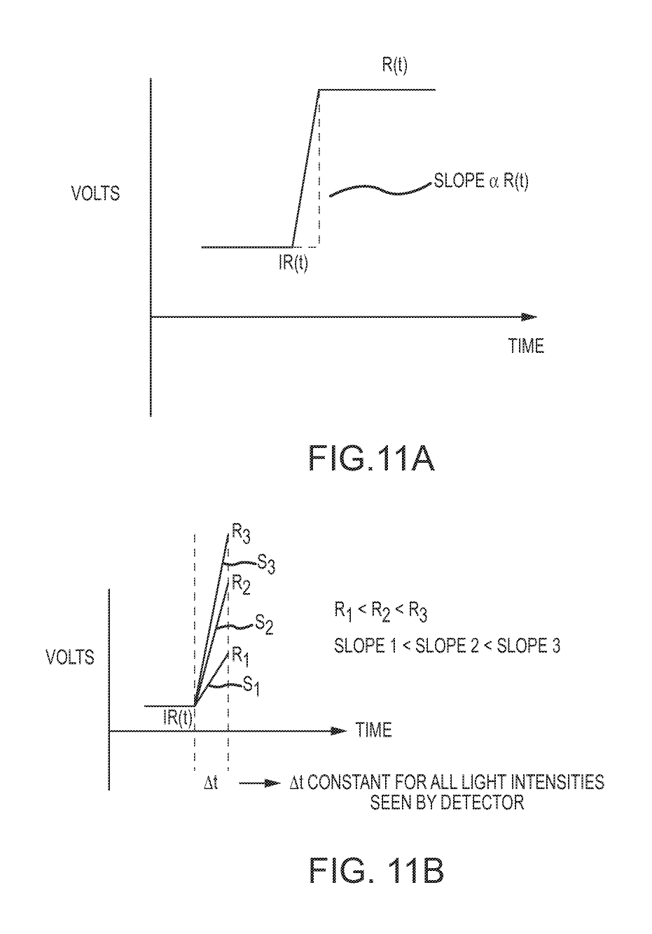

at a specific time window, which may imply the graph illustrated in FIG. 11A. If the turn on time is the same for all levels of light, then relationship shown in FIG. 11B can be obtained, in accordance with various aspects of the subject technology. The foregoing relationship reminds us that

$$\frac{{R}_{max}^{′}}{{IR}_{min}^{′}}$$

is proportional to SpO₂, but since SpO₂ may be proportional to

$$\frac{R ( t )}{IR ( t )} or \frac{{R}_{max}}{{IR}_{max}} ,$$

and equations (1), (2), (3), (4), (5), (6), and/or (7) may be a complicated function of R and IR, it is not obvious how the relationship of

$$\frac{{R}_{max}^{′}}{{IR}_{min}^{′}}$$

can be obtained. Since aspects of the subject technology show that R′/IR′ may provide a function proportional to SpO₂, this relationship may imply that the rising slope may be a strong function of R (see, e.g., FIG. 11A), and similarly, the falling edge may be a strong function of IR. One possible explanation of why R and IR can figure so prominently in the slope is that if the turn on/off time of the detector/LED system is the same or consistent at turn on/off, then the slopes may be strong functions of the R and IR signals (e.g., FIG. 11B shows an example of the R signal). According to certain aspects, the slope may be a difference of the R and IR signals, so the foregoing explanation may be a first order approximation.

According to certain aspects, numerical smoothing of the data via a running average may be applied to the differentiated signal in the signal processing. This may have a similar effect as integrating the signal, although the square wave may not totally be restored as its corners may be rounded due to numerical diffusion.

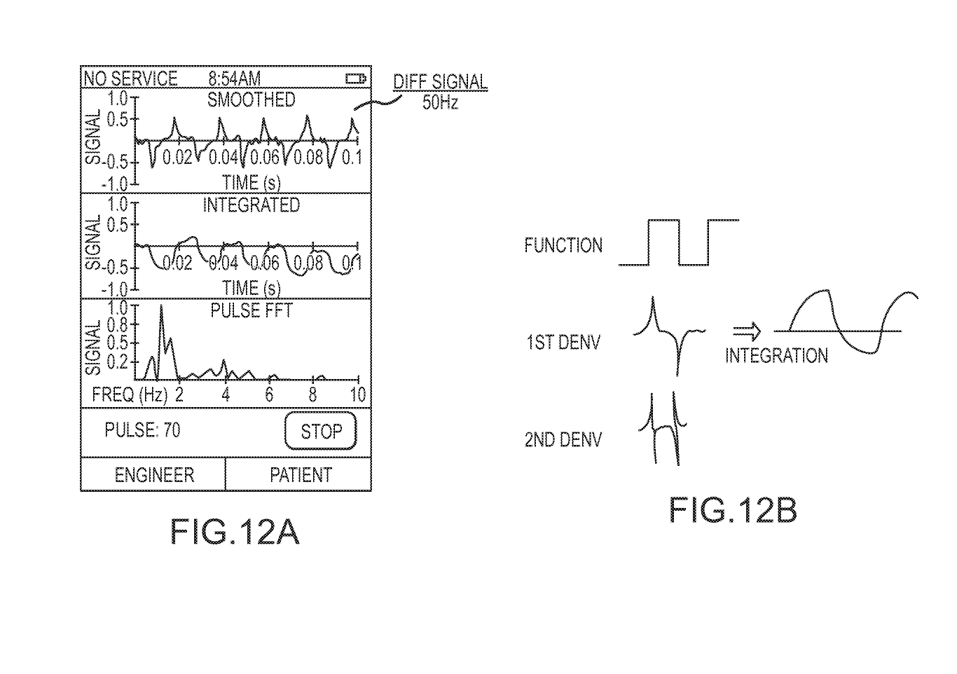

FIGS. 12A and 12B illustrate an example of an alternate scheme to determine SpO₂, in accordance with various aspects of the subject technology. In some aspects, the differentiated signal may be integrated to reconstitute the original square wave. The integration may be performed on each pulse cycle to restore the original square wave. This technique has been tested and shown to be able to determine SpO₂ where the peak max and mins are used (see, e.g., FIG. 12A). The differentiated peak was numerically integrated and the resultant peak shows a rounded square wave (rounding is due to numerical smoothing). Note that the DC offset is not restored in the integration operation.

As shown in FIGS. 12A and 12B, the raw oximeter pulse signal shown (smoothed) and integration of each wave period has been applied to reconstitute the original pre-blocking capacitor waveform which may contain separate red and IR information. This may help in getting more accurate/less noisy pleths, although using the non-integrated signal (e.g., FIGS. 11A and 11B) appears to work in getting SpO₂, pleths, and pulse.

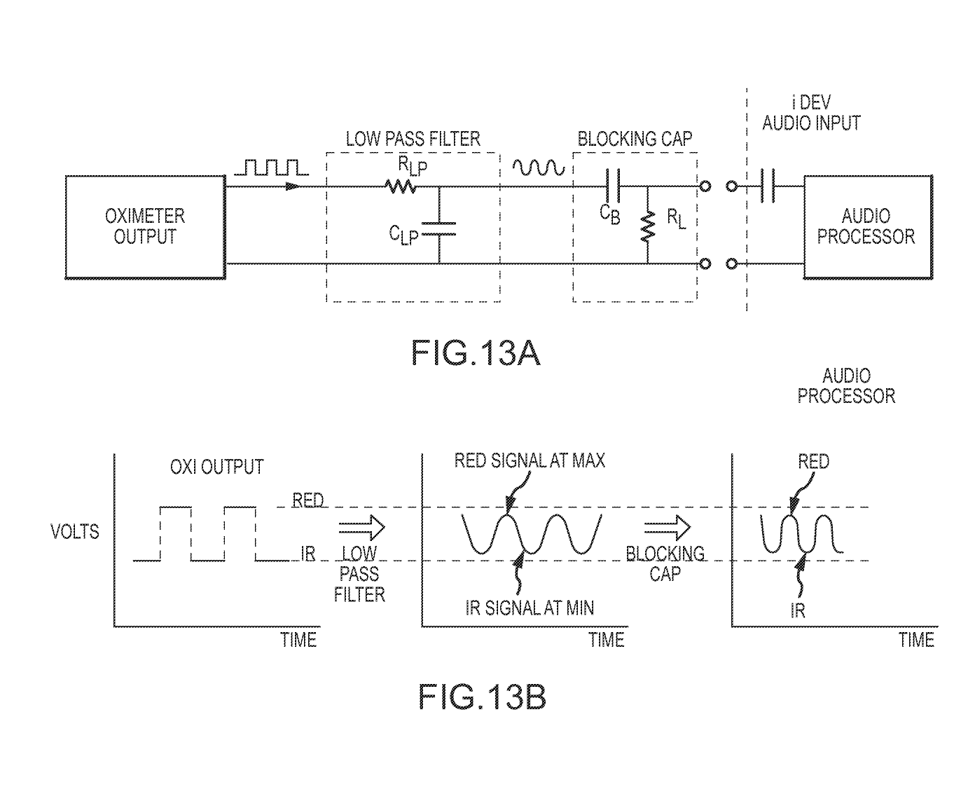

FIGS. 13A and 13B illustrate another example to determine SpO₂, in accordance with various aspects of the subject technology. Instead of dealing with square waves being transformed through the blocking capacitor, it may be possible to send the square wave oximeter output through an electronic low pass filter, then through the blocking capacitor circuit and into the audio port, as shown in FIG. 13A. FIG. 13B illustrates a representation of the signal as it passes through the low pass filter, the blocking capacitor, and into the audio port. According to certain aspects, the low pass filter may be tuned so that the square wave is properly rounded with minimal attenuation so that the resultant waveform may be a sinusoidal wave (or close to sinusoidal). The sinusoidal wave may be transformed into a sine wave with a shifted phase (e.g., cosine) after the blocking capacitor, and if the attenuation is minimized or at least consistent, then the max and min of the cosine wave may be proportional to the R and IR signals respectively. This assumes that the pulse frequency may be fast and that the change in R and IR in each pulse may be minimal. According to certain aspects, at this point, the max and min of the sine waves may be substantially equal or proportional to the initial R and IR signals.

According to certain aspects, using the low pass filter may be equivalent to integrating the signal. Thus, after differentiating the signal through the blocking capacitor, the original signal can be restored (e.g., minus the DC offset). Assuming pulse frequency is sufficiently high such that R(t) in pulse may be constant, then Rₘₐₓ(sine wave) may be proportional or equal to R_{square} and IRₘᵢₙ(sine wave) may be proportional or equal to IR_{square}. This shows the square wave from the oximeter and the resultant sine wave seen by the audio port.

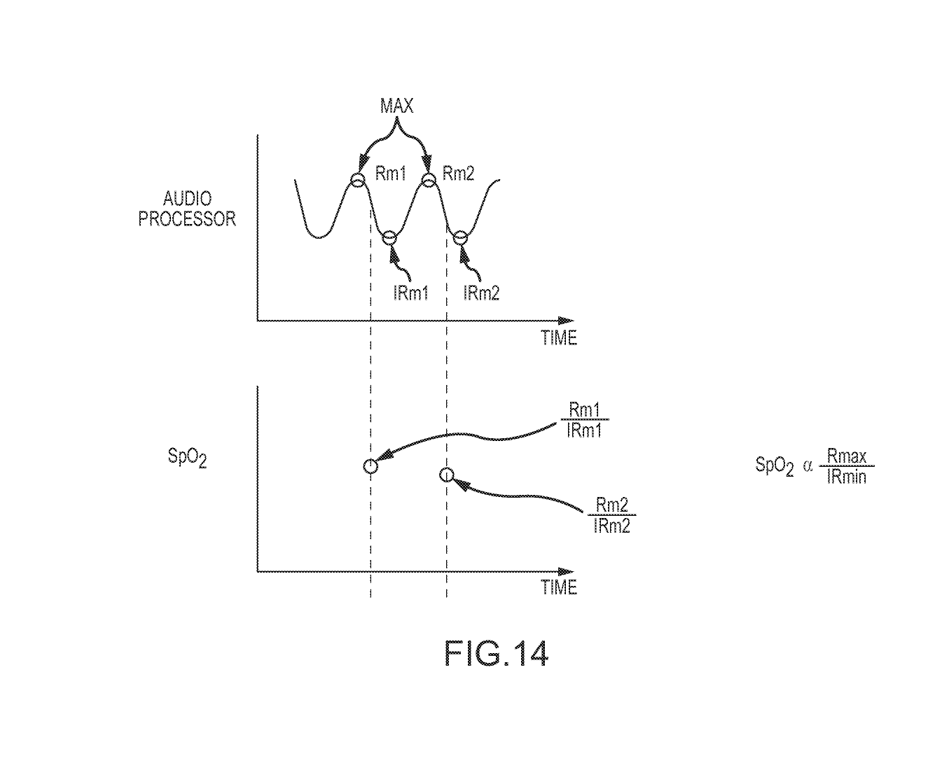

FIG. 14 illustrates an example of how to calculate SpO₂, in accordance with various aspects of the subject technology. In particular, FIG. 14 illustrates how SpO₂ can be calculated from the max and min of the sine wave.

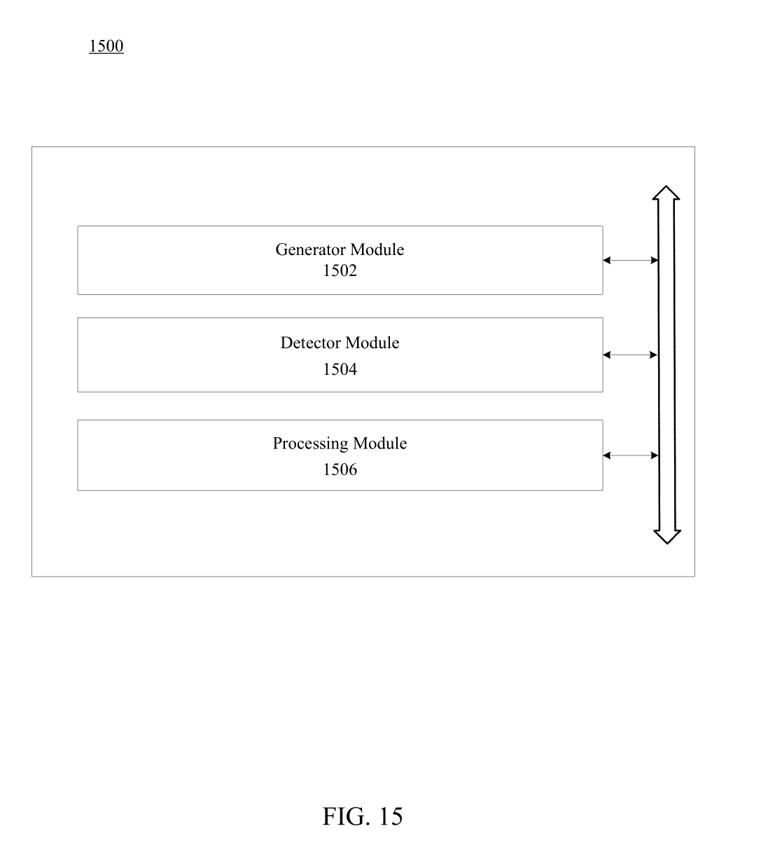

FIG. 15 illustrates an example of system 1500 for estimating SpO₂, in accordance with various aspects of the subject technology. System 1500 comprises generator module 1502, detector module 1504, and processing module 1506. These modules may be in communication with one another. In some aspects, the modules may be implemented in software (e.g., subroutines and code). In some aspects, some or all of the modules may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both.

According to certain aspects, the modules of FIG. 15 may be used to estimate SpO₂ as described herein. In some aspects, generator module 1502 may comprise any component for generating the oximeter output signal (e.g., sensor 110 in FIG. 1, LED drivers 152 in FIG. 1, the oximeter sensor in FIG. 3, the flip flop circuit in FIG. 3, the external battery in FIG. 3, the pulsing hardware in FIG. 4, the oximeter probe in FIG. 5, the amplifier in FIG. 5, the external battery in FIG. 5, the stereo output module in FIG. 5, and/or other suitable components). In some aspects, detector module 1504 may comprise any component for receiving the oximeter output signal (e.g., detector 114 in FIG. 1, signal digitization 154 in FIG. 1, the detector in FIG. 3, one or more of the capacitors in FIG. 3, the load resistor in FIG. 3, the detector in FIG. 5, one or more capacitors in FIG. 5, the load resistor in FIG. 5, and/or other suitable components). In some aspects, processing module 1506 may comprise any component for estimating SpO₂ (e.g., signal processor 156 in FIG. 1, a processor in mobile device 210, a processor in the computer/mobile device in FIG. 3, a processor in the computer/mobile device in FIG. 5, and/or other suitable components). Generator module 1502, detector module 1504, and processing module 1506 may each have one or more components as part of an electronic device (e.g., the computer/mobile device in FIGS. 2, 3, and 5) and/or external to the electronic device.

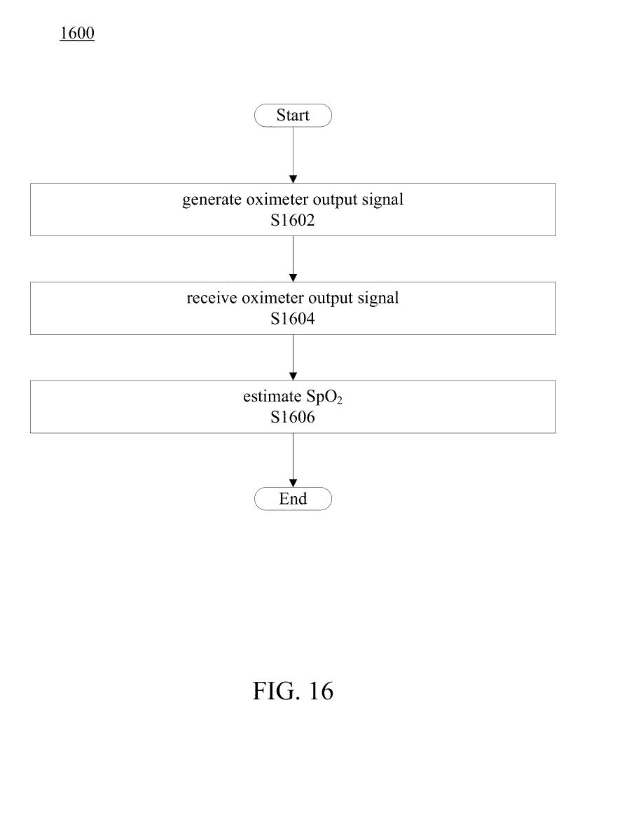

FIG. 16 illustrates an example of method 1600 for estimating SpO₂, in accordance with various aspects of the subject technology. System 1500, for example, may be used to implement method 1600. However, method 1600 may also be implemented by systems having other configurations. Method 1600 may be implemented to estimate SpO₂ as described herein. For example, according to step S1602, generator module 1502 may generate an oximeter output signal. According to step S1604, detector module 1504 may receive the oximeter output signal. According to step S1606, processing module 1506 may estimate SpO₂ based on the oximeter output signal.

According to various aspects of the subject technology, a plethysmographic waveform of a patient (e.g., pulsatile arterial blood flow information of the patient) may also be estimated based on the oximeter output signal. According to certain aspects, the SpO₂ of a patient (e.g., as estimated based on the oximeter output signal) may mirror a plethysmographic waveform of the patient. For example, the estimated SpO₂ and the plethysmographic waveform may be superimposed onto one another. Thus, the plethysmographic waveform may be obtained from the estimated SpO₂.

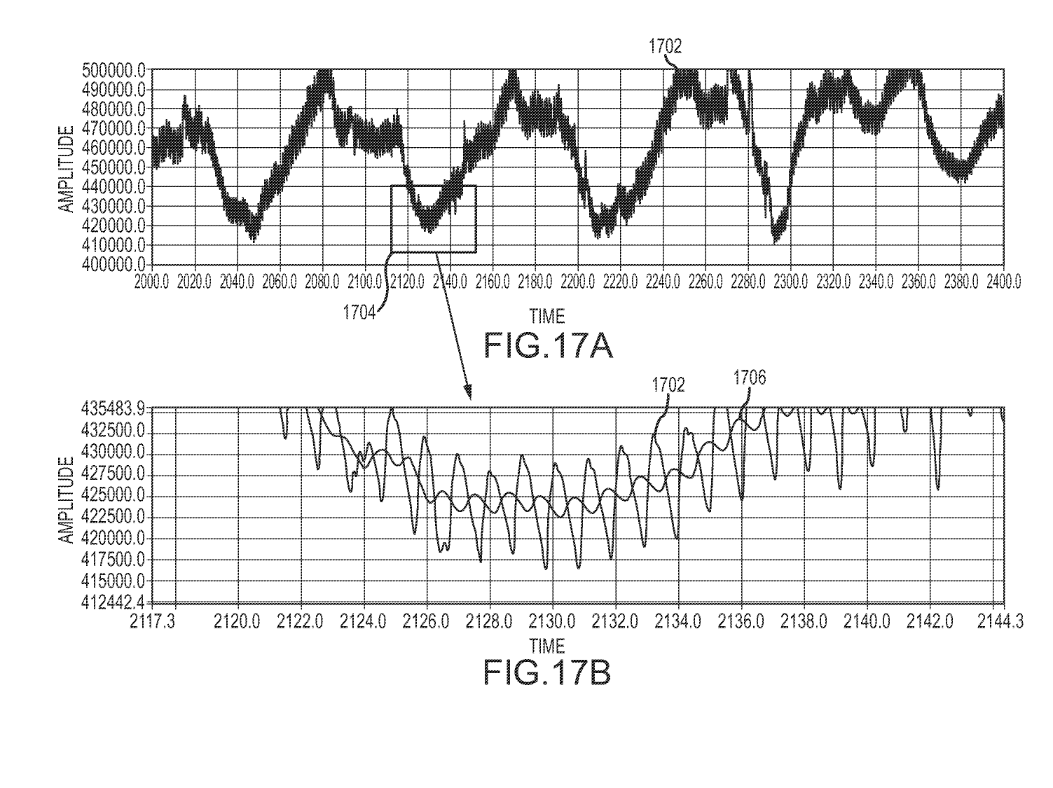

FIGS. 17A and 17B illustrate an example of oximeter output signal 1702 that may be used to determine a plethysmographic waveform of a patient, in accordance with various aspects of the subject technology. In particular, FIG. 17A illustrates a graph of oximeter output signal 1702, with the vertical axis of the graph representing an amplitude of oximeter output signal 1702 and the horizontal axis of the graph representing time (e.g., measured in 20 second intervals). FIG. 17B also illustrates a graph of oximeter output signal 1702, except that the graph in FIG. 17B provides a more detailed view of area 1704 in FIG. 17A. For example, the horizontal axis of the graph in FIG. 17B represents time measured in 2 second intervals. FIG. 17B also illustrates plethysmographic waveform 1706, which substantially follows the curve of oximeter output signal 1702. As shown in FIG. 17B, the changes in plethysmographic waveform 1706 may be small compared to changes in oximeter output signal 1702.

According to certain aspects, oximeter output signal 1702 may be received as described above (e.g., from a single channel that provides alternating red and infrared signals). According to various aspects of the subject technology, an indicator of a ratio of (i) an indicator of the infrared signal to (ii) an indicator of the red signal (or vice versa) may be used to determine plethysmographic waveform 1706. In some aspects, the indicator of the infrared signal may include a derivative, an integral, a peak, a valley (e.g., a minimum such as a local minimum), an average, and/or any other suitable feature of the infrared signal for determining plethysmographic waveform 1706. In some aspects, the indicator of the red signal may include a derivative, an integral, a peak, a valley, an average, and/or any other suitable feature of the red signal for determining plethysmographic waveform 1706. For example, in some aspects, plethysmographic waveform 1706 may be estimated as a ratio of the red signal to the infrared signal. In some aspects, plethysmographic waveform 1706 may be estimated as a ratio of a derivative of the red signal to a derivative of the infrared signal. In some aspects, plethysmographic waveform 1706 may be estimated based on any one or more components of oximeter output signal 1702. For example, according to certain aspects, the red signal and/or the infrared signal may mirror a plethysmographic waveform of a patient. Thus, in accordance with certain aspects, plethysmographic waveform 1706 may be estimated based on a red component, an infrared component, and/or both components of oximeter output signal 1702.

According to various aspects of the subject technology, the heart rate of a patient may also be obtained based on the indicator of the ratio and/or plethysmographic waveform 1706. For example, the heart rate may be obtained based on a frequency of plethysmographic waveform 1706.

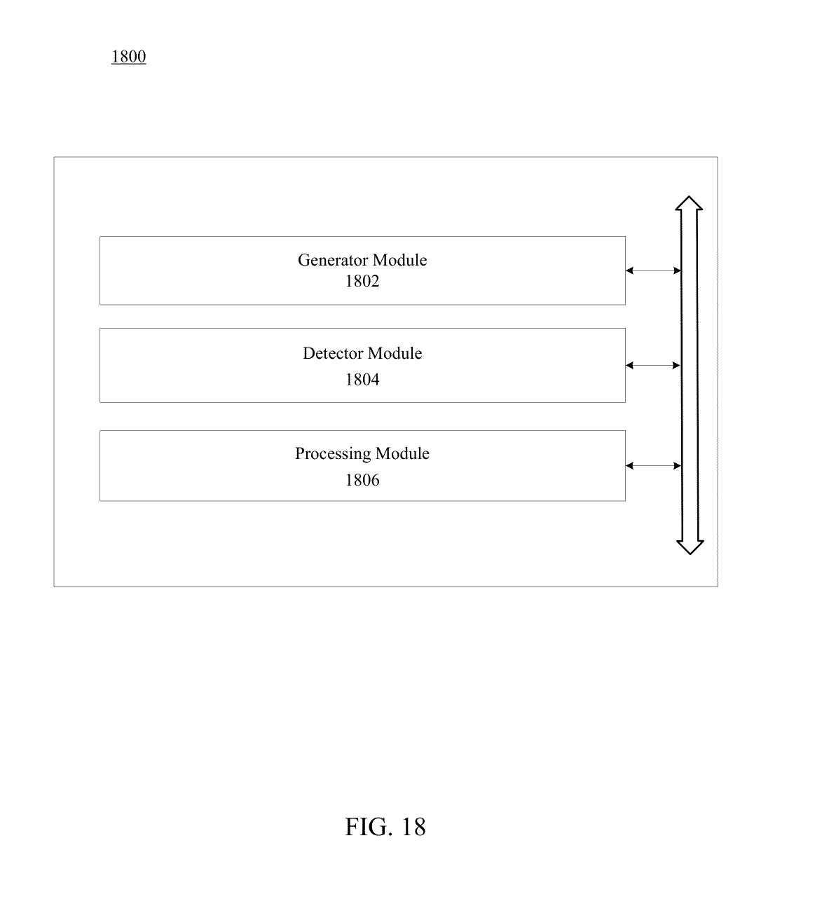

FIG. 18 illustrates an example of system 1800 for estimating a plethysmographic waveform, in accordance with various aspects of the subject technology. System 1800 comprises generator module 1802, detector module 1804, and processing module 1806. These modules may be in communication with one another. In some aspects, the modules may be implemented in software (e.g., subroutines and code). In some aspects, some or all of the modules may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both.

According to certain aspects, the modules of FIG. 18 may be used to estimate a plethysmographic waveform as described herein. In some aspects, generator module 1802 may comprise any component for generating the oximeter output signal (e.g., sensor 110 in FIG. 1, LED drivers 152 in FIG. 1, the oximeter sensor in FIG. 3, the flip flop circuit in FIG. 3, the external battery in FIG. 3, the pulsing hardware in FIG. 4, the oximeter probe in FIG. 5, the amplifier in FIG. 5, the external battery in FIG. 5, the stereo output module in FIG. 5, and/or other suitable components). In some aspects, detector module 1804 may comprise any component for receiving the oximeter output signal (e.g., detector 114 in FIG. 1, signal digitization 154 in FIG. 1, the detector in FIG. 3, one or more of the capacitors in FIG. 3, the load resistor in FIG. 3, the detector in FIG. 5, one or more capacitors in FIG. 5, the load resistor in FIG. 5, and/or other suitable components). In some aspects, processing module 1806 may comprise any component for estimating a plethysmographic waveform (e.g., signal processor 156 in FIG. 1, a processor in mobile device 210, a processor in the computer/mobile device in FIG. 3, a processor in the computer/mobile device in FIG. 5, and/or other suitable components). Generator module 1802, detector module 1804, and processing module 1806 may each have one or more components as part of an electronic device (e.g., the computer/mobile device in FIGS. 2, 3, and 5) and/or external to the electronic device.

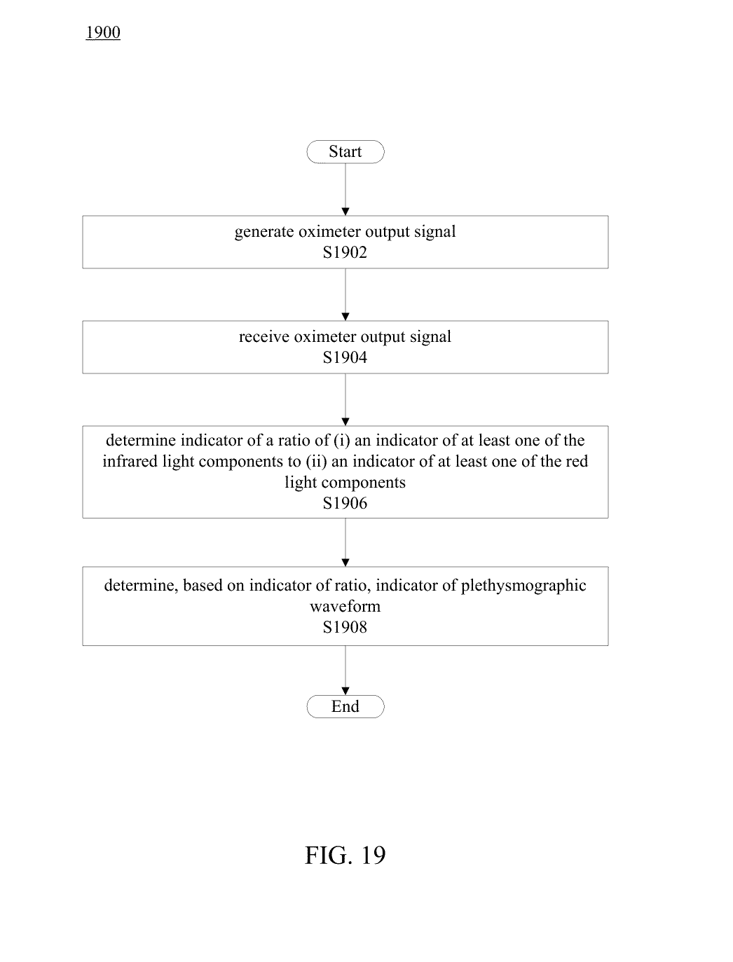

FIG. 19 illustrates an example of method 1900 for estimating a plethysmographic waveform, in accordance with various aspects of the subject technology. System 1800, for example, may be used to implement method 1900. However, method 1900 may also be implemented by systems having other configurations. Method 1900 may be implemented to estimate a plethysmographic waveform as described herein. For example, according to step S1902, generator module 1802 may generate an oximeter output signal. The oximeter output signal may comprise infrared light components (e.g., indicative of infrared light) and red light components (e.g., indicative of red light). According to step S1904, detector module 1804 may receive the oximeter output signal. According to step S1906, processing module 1806 may determine an indicator of a ratio of (i) an indicator of at least one of the infrared light components to (ii) an indicator of at least one of the red light components. According to step S1908, processing module 1806 may determine, based on the indicator of the ratio, an indicator of a plethysmographic waveform.

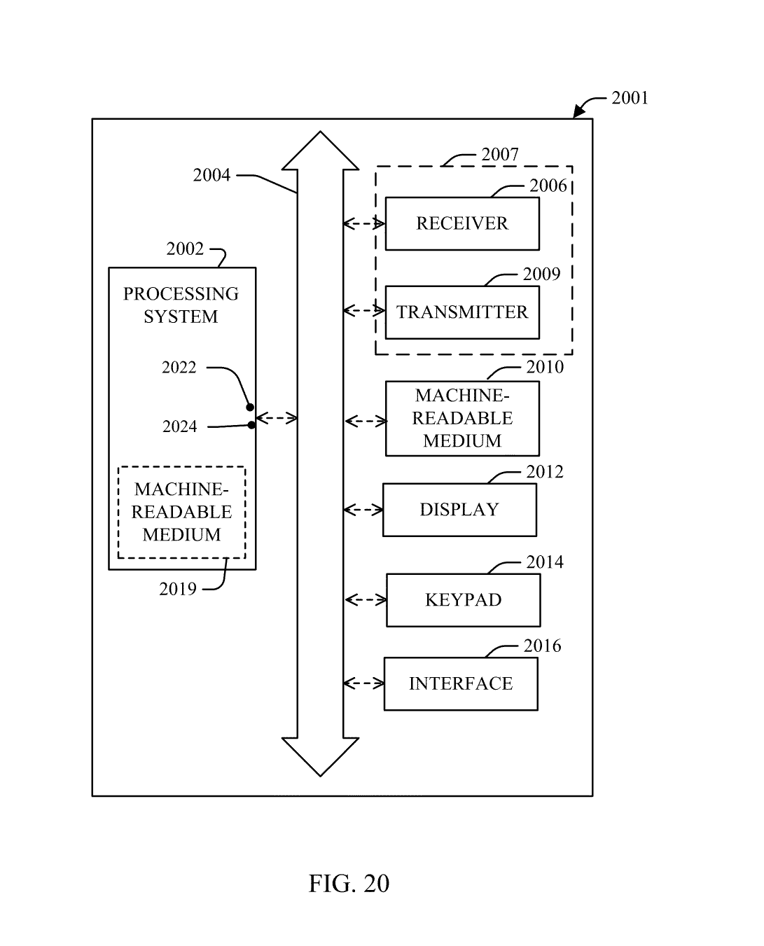

FIG. 20 is a conceptual block diagram illustrating an example of a system, in accordance with various aspects of the subject technology. A system 2001 may be, for example, a client device (e.g., a mobile phone, laptop computer, desktop computer, tablet, or any suitable computing device) or a server. The system 2001 may include a processing system 2002. The processing system 2002 is capable of communication with a receiver 2006 and a transmitter 2009 through a bus 2004 or other structures or devices. It should be understood that communication means other than busses can be utilized with the disclosed configurations. The processing system 2002 can generate audio, video, multimedia, and/or other types of data to be provided to the transmitter 2009 for communication. In addition, audio, video, multimedia, and/or other types of data can be received at the receiver 2006, and processed by the processing system 2002.

The processing system 2002 may include a processor for executing instructions and may further include a machine-readable medium 2019, such as a volatile or non-volatile memory, for storing data and/or instructions for software programs. The instructions, which may be stored in a machine-readable medium 2010 and/or 2019, may be executed by the processing system 2002 to control and manage access to the various networks, as well as provide other communication and processing functions. The instructions may also include instructions executed by the processing system 2002 for various user interface devices, such as a display 2012 and a keypad 2014. The processing system 2002 may include an input port 2022 and an output port 2024. Each of the input port 2022 and the output port 2024 may include one or more ports. The input port 2022 and the output port 2024 may be the same port (e.g., a bi-directional port) or may be different ports.

The processing system 2002 may be implemented using software, hardware, or a combination of both. By way of example, the processing system 2002 may be implemented with one or more processors. A processor may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device that can perform calculations or other manipulations of information.

A machine-readable medium can be one or more machine-readable media. Software shall be construed broadly to mean instructions, data, or any combination thereof, whether referred to as software, firmware, middleware, microcode, hardware description language, or otherwise. Instructions may include code (e.g., in source code format, binary code format, executable code format, or any other suitable format of code).

Machine-readable media (e.g., 2019) may include storage integrated into a processing system, such as might be the case with an ASIC. Machine-readable media (e.g., 2010) may also include storage external to a processing system, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device. Those skilled in the art will recognize how best to implement the described functionality for the processing system 2002. According to certain aspects of the disclosure, a machine-readable medium is a computer-readable medium encoded or stored with instructions and is a computing element, which defines structural and functional interrelationships between the instructions and the rest of the system, which permit the instructions' functionality to be realized. In some aspects, a machine-readable medium is a non-transitory machine-readable medium, a machine-readable storage medium, or a non-transitory machine-readable storage medium. In some aspects, a computer-readable medium is a non-transitory computer-readable medium, a computer-readable storage medium, or a non-transitory computer-readable storage medium. Instructions may be executable, for example, by a client device or server or by a processing system of a client device or server. Instructions can be, for example, a computer program including code.

An interface 2016 may be any type of interface and may reside between any of the components shown in FIG. 20. An interface 2016 may also be, for example, an interface to the outside world (e.g., an Internet network interface). A transceiver block 2007 may represent one or more transceivers, and each transceiver may include a receiver 2006 and a transmitter 2009. A functionality implemented in a processing system 2002 may be implemented in a portion of a receiver 2006, a portion of a transmitter 2009, a portion of a machine-readable medium 2010, a portion of a display 2012, a portion of a keypad 2014, or a portion of an interface 2016, and vice versa.

As used herein, the word “module” refers to logic embodied in hardware or firmware, or to a collection of software instructions, possibly having entry and exit points, written in a programming language, such as, for example C++, Cocoa, an Android-based programming language, and/or other suitable programming languages. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A system, for estimating a plethysmograph waveform, comprising:
a detector module configured to receive, from a single channel, an oximeter output signal indicative of light absorption in a patient, the oximeter output signal comprising infrared light components and red light components; and
a processing module configured to determine an indicator of a ratio of (i) an indicator of at least one of the infrared light components to (ii) an indicator of at least one of the red light components,
wherein the processing module is configured to determine, based on the indicator of the ratio, an indicator of a plethysmograph waveform of the patient.

2. The system of claim 1, wherein the indicator of the at least one red light component comprises at least one of a derivative, an integral, a peak, a valley, or an average of the at least one red light component.

3. The system of claim 1, wherein the indicator of the at least one infrared light component comprises at least one of a derivative, an integral, a peak, a valley, or an average of the at least one infrared light component.

4. The system of claim 1, wherein the indicator of the ratio comprises a saturation level of oxygen in hemoglobin (SpO₂) of the patient.

5. The system of claim 1, wherein the processing module is configured to estimate a heart rate of the patient based on the indicator of the ratio.

6. The system of claim 1, wherein the indicator of the plethysmograph waveform comprises at least one of a heart rate of the patient or pulsatile arterial blood flow information regarding the patient.

7. The system of claim 1, further comprising a generator module configured to generate the oximeter output signal.

8. The system of claim 7, wherein the oximeter output signal alternates between the infrared light components and the red light components.

9. The system of claim 7, wherein the generator module comprises:
a red light module configured to generate the red light components;
an infrared light module configured to generate the infrared light components; and
a driver configured to drive the red light module and the infrared light module such that the red light components and the infrared light components are alternately generated.

10. The system of claim 9, wherein the oximeter output signal comprises the alternately generated red light components and infrared light components.

11. The system of claim 9, wherein the driver is configured to generate a waveform signal that determines which of the red light components and the infrared light components are generated, and wherein the driver is configured to drive the red light module and the infrared light module based on the waveform signal.

12. The system of claim 11, wherein the waveform signal comprises at least one of (i) a headphone output signal from an electronic device or (ii) a stereo output signal from an electronic device.

13. A method, for estimating a plethysmograph waveform, comprising:
receiving, from a single channel, an oximeter output signal indicative of light absorption in a patient, the oximeter output signal comprising infrared light components and red light components;
determining an indicator of a ratio of (i) an indicator of at least one of the infrared light components to (ii) an indicator of at least one of the red light components; and
determining, based on the indicator of the ratio, an indicator of a plethysmograph waveform of the patient.

14. The method of claim 13, wherein the indicator of the at least one red light component comprises at least one of a derivative, an integral, a peak, a valley, or an average of the at least one red light component.

15. The method of claim 13, wherein the indicator of the at least one infrared light component comprises at least one of a derivative, an integral, a peak, a valley, or an average of the at least one infrared light component.

16. The method of claim 13, wherein the indicator of the ratio comprises a saturation level of oxygen in hemoglobin (SpO₂) of the patient.

17. The method of claim 13, further comprising estimating a heart rate of the patient based on the indicator of the ratio.

18. A machine-readable medium encoded with executable instructions for estimating a plethysmograph waveform, the instructions comprising code for:
receiving, from a single channel, an oximeter output signal indicative of light absorption in a patient, the oximeter output signal comprising infrared light components and red light components;
determining an indicator of a ratio of (i) an indicator of at least one of the infrared light components to (ii) an indicator of at least one of the red light components; and
determining, based on the indicator of the ratio, an indicator of a plethysmograph waveform of the patient.

19. The machine-readable medium of claim 18, wherein the indicator of the at least one red light component comprises at least one of a derivative, an integral, a peak, a valley, or an average of the at least one red light component.

20. The machine-readable medium of claim 18, wherein the indicator of the at least one infrared light component comprises at least one of a derivative, an integral, a peak, a valley, or an average of the at least one infrared light component.
